(19) United States
(12) Patent Application Publication
(10) Pub. No.: US 2026/0137317 A1
(43) Pub. Date: May 21, 2026

(54) Blood Sample Management Using Open Cell Foam

(71) Applicant: Becton, Dickinson and Company, Franklin Lakes, NJ (US)

(72) Inventors: Milan Ivosevic, Kinnelon, NJ (US); Alexander James Blake, Ridgewood, NJ (US); Ryan W. Muthard, Wynnewood, PA (US)

(21) Appl. No.: 19/447,679

(22) Filed: Jan. 13, 2026

Related U.S. Application Data
(63) Continuation of application No. 17/691,873, filed on Mar. 10, 2022, now Pat. No. 12,533,061.
Application No. 17/691,873 is a continuation of application No. 14/861,167, filed on Sep. 22, 2015, now Pat. No. 11,298,061.
(60) Provisional application No. 62/063,536, filed on Oct. 14, 2014.
(60) Provisional application No. 62/207,618, filed on Aug. 20, 2015.

(51) Int. Cl. A61B 5/15; A61B 5/151; A61B 10/00; A61J 1/06; A61J 1/14; B01L 3/00; G01N 1/36

(52) U.S. Cl. CPC A61B 5/150755; A61B 5/150022; A61B 5/150221; A61B 5/150236; A61B 5/150244; A61B 5/150259; A61B 5/150305; A61B 5/150343; A61B 5/150351; A61B 5/150366; A61B 5/151; A61B 5/15113; A61B 5/15142; A61J 1/067; A61J 1/1412; A61J 1/1475; B01L 3/502; B01L 3/502707; B01L 3/502746; B01L 3/567; G01N 1/36; A61B 10/0045; B01L 2200/12; B01L 2300/042; B01L 2300/069; B01L 2300/0838; B01L 2300/0858; B01L 2300/12; B01L 2300/123; B01L 2300/16; B01L 2400/0478; B01L 2400/06; B01L 2400/08; B01L 2400/086; B01L 2400/088

(57) ABSTRACT

A specimen mixing and transfer device adapted to receive a sample is disclosed. The specimen mixing and transfer device includes a housing, a material including pores that is disposed within the housing, and a dry anticoagulant powder within the pores of the material. In one embodiment, the material is a sponge material. In other embodiments, the material is an open cell foam. In one embodiment, the material is treated with an anticoagulant to form a dry anticoagulant powder finely distributed throughout the pores of the material. A blood sample may be received within the specimen mixing and transfer device. The blood sample is exposed to and mixes with the anticoagulant powder while passing through the material.

### CROSS-REFERENCE TO RELATED APPLICATIONS

This application is a continuation of U.S. application Ser. No. 17/691,873, entitled “Blood Sample Management Using Open Cell Foam” filed on Mar. 10, 2022, which is a continuation of U.S. application Ser. No. 14/861,167, entitled “Blood Sample Management Using Open Cell Foam” filed on Sep. 22, 2015 (now U.S. Pat. No. 11,298,061), which claims priority to U.S. Provisional Application Ser. No. 62/207,618, entitled “Blood Sample Management Using Open Cell Foam” filed Aug. 20, 2015, and U.S. Provisional Application Ser. No. 62/063,536, entitled “Blood Sample Management Using Open Cell Foam” filed Oct. 14, 2014, and, the entire disclosures of each of which are herein incorporated by reference.

### BACKGROUND OF THE INVENTION

### 1. Field of the Disclosure

The present disclosure relates generally to a blood transfer device. More particularly, the present disclosure relates to a blood transfer device, a blood transfer and testing system, a lancet and blood transfer device, and a method of loading an anticoagulant.

### 2. Description of the Related Art

Blood sampling is a common health care procedure involving the withdrawal of at least a drop of blood from a patient. Blood samples are commonly taken from hospitalized, homecare, and emergency room patients either by finger stick, heel stick, or venipuncture. Once collected, blood samples may be analyzed to obtain medically useful information including, for example, chemical composition, hematology, and coagulation.

Blood tests determine the physiological and biochemical states of the patient, such as disease, mineral content, drug effectiveness, and organ function. Blood tests may be performed in a clinical laboratory or at the point-of-care near the patient.

### SUMMARY OF THE INVENTION

The present disclosure provides a specimen mixing and transfer device adapted to receive a sample. The specimen mixing and transfer device includes a housing, a material including pores that is disposed within the housing, and a dry anticoagulant powder within the pores of the material. In one embodiment, the material is a sponge material. In other embodiments, the material is an open cell foam. In one embodiment, the open cell foam is treated with an anticoagulant to form a dry anticoagulant powder finely distributed throughout the pores of the material. A blood sample may be received within the specimen mixing and transfer device. The blood sample is exposed to and mixes with the anticoagulant powder while passing through the material.

A specimen mixing and transfer device of the present disclosure offers uniform and passive blood mixing with an anticoagulant under flow-through conditions. A specimen mixing and transfer device of the present disclosure could catch blood clots or other contaminants within the microstructure of the material and prevent them from being dispensed into a diagnostic sample port. A specimen mixing and transfer device of the present disclosure enables a simple, low-cost design for passive flow-through blood stabilization. A specimen mixing and transfer device of the present disclosure enables precisely controlled loading of an anticoagulant into the material by soaking it with an anticoagulant and water solution and then drying the material to form a finely distributed dry anticoagulant powder throughout the pores of the material.

A specimen mixing and transfer device of the present disclosure may provide an effective passive blood mixing solution for applications wherein blood flows through a line. Such a specimen mixing and transfer device is useful for small blood volumes, e.g., less than 50 μL or less than 500 μL, and/or where inertial, e.g., gravity based, forces are ineffective for bulk manual mixing by flipping back and forth a blood collection container such as is required for vacuum tubes.

In accordance with an embodiment of the present invention, a specimen mixing and transfer device adapted to receive a sample includes a housing having a first end, a second end, and a sidewall extending therebetween; a material including pores and disposed within the housing; and a dry anticoagulant powder within the pores of the material.

In one configuration, the sample is a blood sample. In another configuration, the housing is adapted to receive the blood sample therein via the first end. In yet another configuration, with the blood sample received within the housing, the blood sample passes through the material thereby effectively mixing the blood sample with the dry anticoagulant powder. In one configuration, the blood sample dissolves and mixes with the dry anticoagulant powder while passing through the material. In another configuration, the material is an open cell foam. In yet another configuration, the material is a sponge. In one configuration, the first end includes an inlet. In another configuration, the second end includes an outlet. In yet another configuration, the housing defines a mixing chamber having a material including pores disposed within the mixing chamber. In one configuration, the housing includes an inlet channel in fluid communication with the inlet and the mixing chamber and an outlet channel in fluid communication with the mixing chamber and the outlet. In another configuration, the housing includes a dispensing chamber between the mixing chamber and the outlet.

In accordance with another embodiment of the present invention, a specimen mixing and transfer device adapted to receive a sample includes a housing having a first end, a second end, and a sidewall extending therebetween; a dry anticoagulant powder disposed within the housing; and a mixing element disposed within the housing.

In one configuration, the sample is a blood sample. In another configuration, the housing is adapted to receive the blood sample therein via the first end. In yet another configuration, with the blood sample received within the housing, the mixing element interferes with a flow of the blood sample to promote mixing of the blood sample with the dry anticoagulant powder. In one configuration, the dry anticoagulant powder is deposited on an interior surface of the housing. In another configuration, the mixing element comprises a plurality of posts. In one configuration, the first end includes an inlet. In another configuration, the second end includes an outlet. In yet another configuration, the housing defines a mixing chamber having a dry anticoagulant powder disposed within the mixing chamber. In one configuration, the housing includes an inlet channel in fluid communication with the inlet and the mixing chamber and an outlet channel in fluid communication with the mixing chamber and the outlet. In another configuration, the housing includes a dispensing chamber between the mixing chamber and the outlet. In yet another configuration, the housing includes two diverted flow channels between the inlet channel and the outlet channel.

In accordance with yet another embodiment of the present invention, a method of loading an anticoagulant to a material having pores includes soaking the material in a liquid solution of the anticoagulant and water; evaporating the water of the liquid solution; and forming a dry anticoagulant powder within the pores of the material.

In one configuration, the material is a sponge. In another configuration, the material is an open cell foam.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a partial cross-sectional view of a specimen mixing and transfer device in accordance with an embodiment of the present invention.

FIG. 2 is a microscopic view of the microstructure of an open cell foam material having a dry anticoagulant powder distributed throughout its microstructure in accordance with an embodiment of the present invention.

FIG. 3 is a partial cross-sectional view of a specimen mixing and transfer device in accordance with another embodiment of the present invention.

FIG. 4 is a perspective view of a specimen mixing and transfer device in accordance with an embodiment of the present invention.

FIG. 5 is a partial cross-sectional view of a specimen mixing and transfer device in accordance with an embodiment of the present invention.

FIG. 6 is a partial cross-sectional view taken along line 6-6 of FIG. 5 in accordance with an embodiment of the present invention.

FIG. 7 is a perspective view of a specimen mixing and transfer device in accordance with another embodiment of the present invention.

FIG. 8 is a partial cross-sectional view of a specimen mixing and transfer device in accordance with another embodiment of the present invention.

FIG. 9 is a partial cross-sectional view taken along line 9-9 of FIG. 8 in accordance with an embodiment of the present invention.

FIG. 10 is a perspective view of alternate embodiments of a specimen mixing and transfer device in accordance with another embodiment of the present invention.

FIG. 11A is a perspective view of a syringe assembly in accordance with an embodiment of the present invention.

FIG. 11B is a close-up partial perspective view of the syringe assembly of FIG. 11A in accordance with an embodiment of the present invention.

FIG. 11C is a perspective view of a syringe assembly in accordance with an embodiment of the present invention.

FIG. 12 is a perspective view of an open cell foam material in accordance with an embodiment of the present invention.

FIG. 13 is a microscopic view of the microstructure of an open cell foam material having a dry anticoagulant powder distributed throughout its microstructure in accordance with an embodiment of the present invention.

FIG. 14 is a microscopic view of the microstructure of an untreated foam material.

FIG. 15 is a perspective view of a syringe assembly in accordance with an embodiment of the present invention.

FIG. 16 is a graph demonstrating the anticoagulant uptake by a blood sample flowing through an open cell foam material having a dry anticoagulant powder distributed throughout its microstructure in accordance with an embodiment of the present invention.

FIG. 17 is a perspective view of a blood transfer system in accordance with an embodiment of the present invention.

FIG. 18 is a perspective view of a blood transfer system in accordance with an embodiment of the present invention.

FIG. 19 is a perspective view of a blood transfer system in accordance with an embodiment of the present invention.

FIG. 20 is a perspective view of a blood transfer system in accordance with an embodiment of the present invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

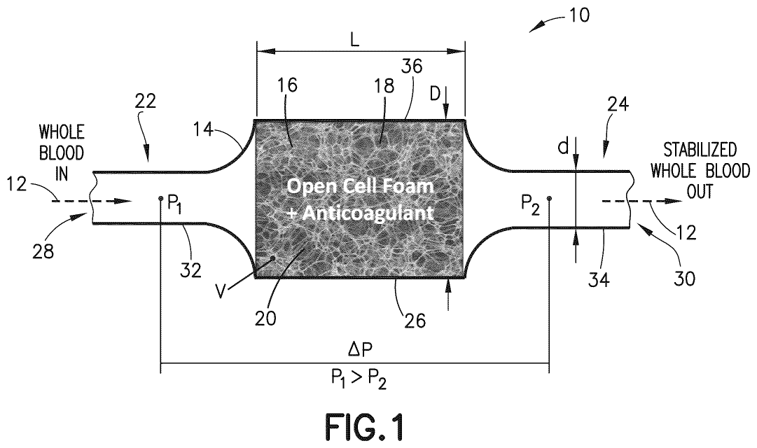

FIGS. 1-3 illustrate exemplary embodiments of a specimen mixing and transfer device of the present disclosure. The specimen mixing and transfer device 10 is adapted to receive a sample 12. In one embodiment, the specimen mixing and transfer device 10 includes a housing 14, a material 16 including pores 18 that is disposed within the housing 14, and a dry anticoagulant powder 20 within the pores 18 of the material 16.

With a sample 12 received within the specimen mixing and transfer device 10, a portion of the specimen mixing and transfer device 10 acts as a flow-through chamber for the effective mixing of a sample 12 with the dry anticoagulant powder 20 within the material 16. In other embodiments, the material 16 may contain other dry substances. The effective mixing is achieved by passing the sample 12 through the material 16 having the dry anticoagulant powder 20 distributed throughout its microstructure.

A specimen mixing and transfer device 10 of the present disclosure offers uniform and passive blood mixing with an anticoagulant under flow-through conditions. A specimen mixing and transfer device 10 of the present disclosure may catch blood clots or other contaminants within the microstructure of the material 16 and prevent them from being dispensed into a diagnostic sample port. A specimen mixing and transfer device 10 of the present disclosure enables a simple, low cost design for passive flow-through blood stabilization. A specimen mixing and transfer device 10 of the present disclosure enables precisely controlled loading of an anticoagulant into the material 16 by soaking it with an anticoagulant and water solution and then drying the material 16 to form a finely distributed dry anticoagulant powder 20 throughout the pores 18 of the material 16.

A specimen mixing and transfer device 10 of the present disclosure may provide an effective passive blood mixing solution for applications wherein blood flows through a line. Such a specimen mixing and transfer device 10 is useful for small blood volumes, e.g., less than 50 μL or less than 500 μL, and/or where inertial, e.g., gravity based, forces are ineffective for bulk manual mixing by flipping back and forth a blood collection container such as is required for vacuum tubes.

FIG. 1 illustrates an exemplary embodiment of a specimen mixing and transfer device 10 of the present disclosure. Referring to FIG. 1, in one embodiment, a specimen mixing and transfer device 10 includes a housing 14, a material 16 including pores 18 that are disposed within the housing 14, and a dry anticoagulant powder 20 within the pores 18 of the material 16. The housing 14 includes a first end 22, a second end 24, and a sidewall 26 extending between the first end 22 and the second end 24. In one embodiment, the first end 22 includes an inlet 28 and the second end 24 includes an outlet 30.

Referring to FIG. 1, in one embodiment, the housing 14 of the specimen mixing and transfer device 10 includes an inlet channel 32 and an outlet channel 34. The inlet channel 32 and the outlet channel 34 are in fluid communication via a flow channel or mixing chamber 36. For example, the inlet channel 32 is in fluid communication with the inlet 28 and the mixing chamber 36; and the outlet channel 34 is in fluid communication with the mixing chamber 36 and the outlet 30. In one embodiment, the material 16 is disposed within the mixing chamber 36 of the housing 14.

In one embodiment, the material 16 is a sponge material. In other embodiments, the material 16 is an open cell foam. In one embodiment, the open cell foam is treated with an anticoagulant, as described in detail below, to form a dry anticoagulant powder 20 finely distributed throughout the pores 18 of the material 16. A sample 12 may be received within the specimen mixing and transfer device 10. In some embodiments, the sample 12 gets soaked into the material 16 based on capillary principles. In some embodiments, the sample 12 may be a blood sample. The blood sample is exposed to and mixes with the anticoagulant powder 20 while passing through the intricate microstructure of the material 16. In this manner, the specimen mixing and transfer device 10 produces a stabilized sample. In some embodiments, the stabilized sample may be transferred to a diagnostic instrument such as a blood testing device, a point-of-care testing device, or similar analytical device.

In one embodiment, the material 16 is an open cell foam. For example, the material 16 is a soft deformable open cell foam that is inert to blood. In one embodiment, the open cell foam may be a melamine foam, such as Basotect® foam commercially available from BASF. In another embodiment, the open cell foam may consist of a formaldehyde-melamine-sodium bisulfite copolymer. The open cell foam may be a flexible, hydrophilic open cell foam that is resistant to heat and many organic solvents. In one embodiment, the open cell foam may be a sponge material.

A method of loading an anticoagulant to a material 16 having pores 18 will now be discussed. In one embodiment, the method includes soaking the material 16 in a liquid solution of the anticoagulant and water; evaporating the water of the liquid solution; and forming a dry anticoagulant powder 20 within the pores 18 of the material 16.

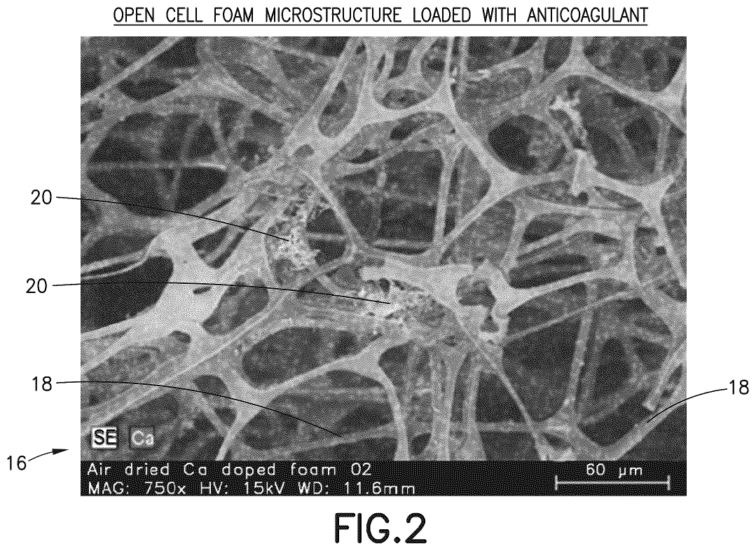

The method of the present disclosure enables precisely controlled loading of an anticoagulant into the material 16 by soaking it with an anticoagulant and water solution and then drying the material 16 to form a finely distributed dry anticoagulant powder 20 throughout the pores 18 of the material 16, as shown in FIG. 2.

Anticoagulants such as Heparin or EDTA (Ethylene Diamine Tetra Acetic Acid), as well as other blood stabilization agents, could be introduced into the material 16 as a liquid solution by soaking the material 16 in the liquid solution of a desired concentration. After evaporating the liquid phase, e.g., evaporating the water from a water and Heparin solution, a dry anticoagulant powder 20 is formed and finely distributed throughout the internal structure of the material 16, as shown in FIG. 2. For example, the dry anticoagulant powder 20 is formed and finely distributed throughout the pores 18 of the material 16. In a similar manner, the material 16 could be treated to provide a hydrophobic, hydrophilic, or reactive internal pore surface.

In one configuration, a key advantage of providing an open cell foam as the material 16 is that a known amount of anticoagulant may be loaded into the pores 18 of the foam material. A desired concentration of an anticoagulant may be dissolved in water or other suitable solvent and then introduced into the pores 18 of the open cell foam material 16 in liquid form. In one embodiment, the anticoagulant may be loaded into the pores 18 by dipping the open cell foam material 16 into a solution of anticoagulant and water or solvent and subsequently allowing the open cell foam material 16 to dry. The open cell foam material 16 may be allowed to dry in ambient air or in a heated oven. After drying, the anticoagulant may be distributed throughout the internal microstructure of the open cell foam material 16 in the form of a dry powder.

It is noted that suitable hydrophilic foam material having interconnected cell pores may be loaded with anticoagulant, as described above, and used as described herein for flow-through blood stabilization.

One key advantage of using a melamine-based open cell foam material is that melamine foams have a generally low analyte bias. As discussed herein, analyte bias is the difference in a measured value of an analyte as compared to a blood control value. Generally, analyte bias occurs when analytes adhere to a surface of a material, when analytes are leached from a material, via introduction of other components which may interfere with a measurement, or upon activation of a biological process. Additional open cell foam materials which are suitable for use as described herein include organic thermoplastic and thermosetting polymers and co-polymers, including but not limited to polyolefins, polyimides, polyamides, such as polyethylene terephthalate (PET), polypropylene (PP), polyethylene (PE), and the like. The material may be in fibrous structure, such as woven or random fiber form, or irregular 3D structure.

In order to avoid or minimize potential analyte bias associated with the housing 14 of the transfer device 10, the material of the housing 14 may be treated. In one embodiment, the housing 14 may be treated with an additive coating which acts to block analytes from sticking to a surface. Additive coatings may include, but are not limited to, 1.) proteins, such as bovine serum albumin (BSA), casein, or non-fat milk, 2.) surfactants such as polysorbate 20 (Tween 20) and organosilicone (L-720), 3.) polymers and copolymers such as polyethylene glycol (PEG), polyvinyl alcohol (PVA), and polyvinylpyrrolidone (PVP), 4.) carbohydrates such as destran and glycosamino glycans, such as heparin, and 5.) cell membrane mimicking polymers such as Lipidure.

Alternatively, the housing 14 may be treated with a chemical surface modification. Chemical surface modifications can include, but are not limited to, 1.) gas plasma treatment, 2.) chemical bonding or polyethylene glycol (PEG) or other polymers to achieve a desired hydrophobicity or hydrophilicity, 3.) chemical modification of the surface to include hydrophilic compositions such as ethylene glycol, or hydrophobic groups, such as long carbon chains, and 4.) vapor deposition of a substance, such as parylene. It is appreciated herein that combinations of any of the above materials may be used to achieve the desired properties to minimize analyte bias for a specific analyte or group of analytes.

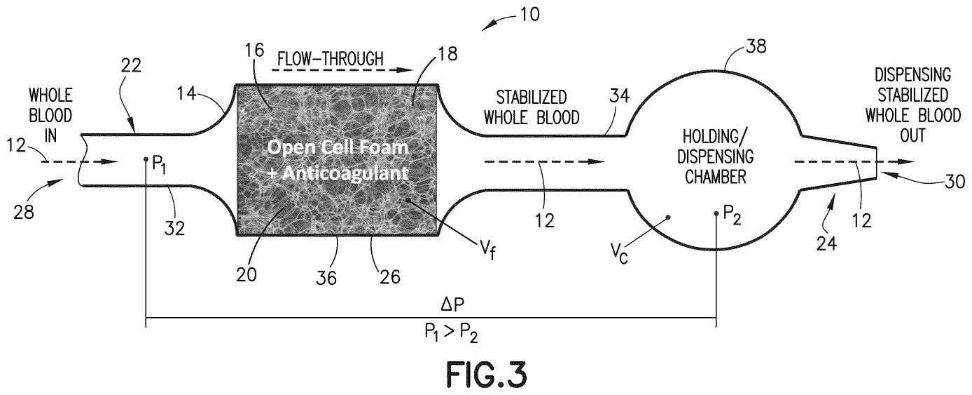

In one embodiment, the mixing chamber 36 includes the material 16 having a dry anticoagulant powder 20 therein. For example, referring to FIGS. 1 and 3, the material 16 is disposed within the mixing chamber 36 of the specimen mixing and transfer device 10. The anticoagulant can be loaded into the material 16 having pores 18 as described above.

Referring to FIG. 1, the housing 14 of the specimen mixing and transfer device 10 is adapted to receive a sample 12 therein via the first end 22. For example, the housing 14 of the specimen mixing and transfer device 10 is adapted to receive a sample 12 therein via the inlet 28. After the sample 12 enters the specimen mixing and transfer device 10 via the inlet 28, the sample 12 flows through the inlet channel 32 to the mixing chamber 36.

With the sample 12 received within the mixing chamber 36, the mixing chamber 36 acts as a flow-through chamber for the effective mixing of a sample 12 with the dry anticoagulant powder 20 within the material 16. In other embodiments, the material 16 may contain other dry substances. The effective mixing is achieved by passing the sample 12 through the material 16 having the dry anticoagulant powder 20 distributed throughout its microstructure. The sample 12 dissolves and mixes with the dry anticoagulant powder 20 while passing through the material 16.

Referring to FIG. 2, a view of the microstructure of the material 16 having a dry anticoagulant powder 20 distributed throughout its microstructure, e.g., its pores 18, is illustrated.

Referring to FIG. 3, in one embodiment, the housing 14 of the specimen mixing and transfer device 10 includes a dispensing chamber or holding chamber 38. The dispensing chamber 38 may be adjacent the outlet 30 of the specimen mixing and transfer device 10. For example, the dispensing chamber 38 may be disposed between the mixing chamber 36 and the outlet 30.

After the blood sample is exposed to and mixes with the anticoagulant powder 20 while passing through the intricate microstructure of the material 16, a stabilized sample flows from the material 16 to the dispensing chamber 38 via the outlet channel 34. The stabilized sample can remain within the dispensing chamber 38 until it is desired to transfer the stabilized sample from the specimen mixing and transfer device 10. For example, the stabilized sample may be transferred to a diagnostic instrument such as a blood testing device, a point-of-care testing device, or similar analytical device.

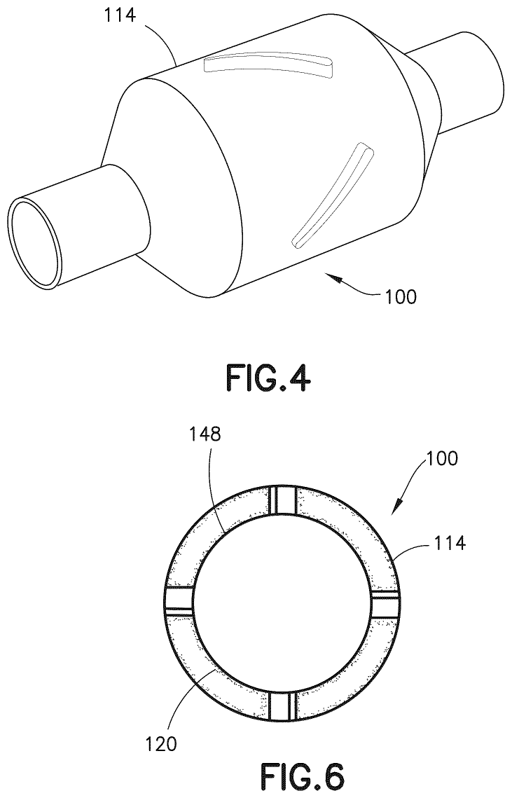

FIGS. 4-10 illustrate other exemplary embodiments of a specimen mixing and transfer device of the present disclosure. Referring to FIGS. 4-10, a specimen mixing and transfer device of the present disclosure may also be effective with small blood volumes that are typically associated with laminar flow conditions that require flow obstacles to promote mixing with a dry anticoagulant deposited on the walls of the flow-through structure.

FIGS. 4-6 illustrate another exemplary embodiment of a specimen mixing and transfer device of the present disclosure. The specimen mixing and transfer device 100 is adapted to receive a sample 112. In some embodiments, the sample 112 may be a blood sample. In one embodiment, the specimen mixing and transfer device 100 includes a housing 114, a dry anticoagulant powder 120 disposed within the housing 114, and a mixing element 115 disposed within the housing 114.

The housing 114 includes a first end 122, a second end 124, and a sidewall 126 extending between the first end 122 and the second end 124. In one embodiment, the first end 122 includes an inlet 128 and the second end 124 includes an outlet 130.

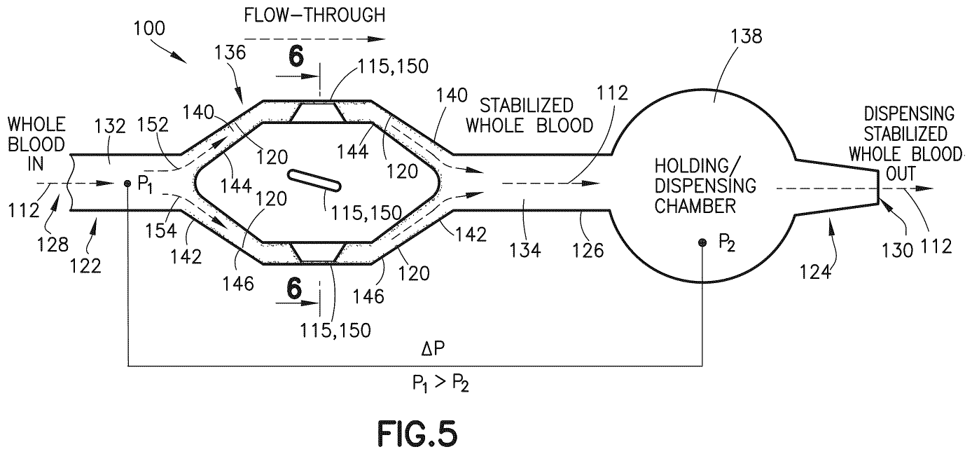

Referring to FIG. 5, in one embodiment, the housing 114 of the specimen mixing and transfer device 100 includes an inlet channel 132 and an outlet channel 134. The inlet channel 132 and the outlet channel 134 are in fluid communication via a flow channel or mixing chamber 136. For example, the inlet channel 132 is in fluid communication with the inlet 128 and the mixing chamber 136; and the outlet channel 134 is in fluid communication with the mixing chamber 136 and the outlet 130. In one embodiment, the dry anticoagulant powder 120 is disposed within the mixing chamber 136 of the housing 114.

In one embodiment, the inlet channel 132 and the outlet channel 134 are in fluid communication via a first flow channel 140 and a second flow channel 142. For example, the inlet channel 132 may branch off into two separate flow channels, e.g., the first flow channel 140 and the second flow channel 142. The two separate flow channels, e.g., the first flow channel 140 and the second flow channel 142, may both flow into the outlet channel 134 as shown in FIG. 5.

The first flow channel 140 includes walls 144 and the second flow channel 142 includes walls 146. In one embodiment, a first portion of the dry anticoagulant powder 120 is deposited on walls 144 and a second portion of the dry anticoagulant powder 120 is deposited on walls 146. For example, in one embodiment, a first portion of the dry anticoagulant powder 120 is deposited on an interior surface 148 of the housing 114, e.g., an interior surface of wall 144, and a second portion of the dry anticoagulant powder 120 is deposited on an interior surface 148 of the housing 114, e.g., an interior surface of wall 146.

Referring to FIG. 5, in one embodiment, the housing 114 of the specimen mixing and transfer device 100 includes a dispensing chamber or holding chamber 138. The dispensing chamber 138 may be adjacent to the outlet 130 of the specimen mixing and transfer device 100. For example, the dispensing chamber 138 may be disposed between the mixing chamber 136 and the outlet 130. In one embodiment, the dispensing chamber 138 may be positioned between the flow channels 140, 142 and the outlet 130.

In one embodiment, the specimen mixing and transfer device 100 includes a mixing element 115 disposed within the housing 114. For example, a portion of the mixing chamber 136 may also include obstacles or mixing promoters 150 that interfere with the flow path of the blood sample thereby promoting mixing between the blood sample and the dry anticoagulant powder 120. In some embodiments, a portion of the first flow channel 140 and a portion of the second flow channel 142 may include obstacles or mixing promoters 150 that interfere with the flow path of the blood sample thereby promoting mixing between the blood sample and the dry anticoagulant powder 120.

Referring to FIGS. 4-6, the specimen mixing and transfer device 100 is adapted to receive a sample 112 therein via the first end 122. For example, the housing 114 of the specimen mixing and transfer device 100 is adapted to receive a sample 112 therein via the inlet 128. The sample 112 flows into the inlet 128 and to the inlet channel 132. In some embodiments, the sample 112 may be a blood sample.

With the blood sample received within the inlet channel 132, a first portion 152 of the blood sample flows to the first flow channel 140 and a second portion 154 of the blood sample flows to the second flow channel 142. The first flow channel 140 provides a first flow path for the first portion 152 of the blood sample and the second flow channel 142 provides a second flow path for the second portion 154 of the blood sample.

With the first portion 152 of the blood sample received within the first flow channel 140, the first portion 152 of the blood sample mixes with a first portion of the dry anticoagulant powder 120 deposited on the walls 144 of the first flow channel 140. The first flow channel 140 may also include obstacles or mixing promoters 150 that interfere with the flow path of the blood sample thereby promoting mixing between the blood sample and the first portion of the dry anticoagulant powder 120. After mixing, the first portion 152 of the blood sample and the first portion of the dry anticoagulant powder 120, i.e., a stabilized blood sample, travel to the outlet channel 134.

With the second portion 154 of the blood sample received within the second flow channel 142, the second portion 154 of the blood sample mixes with a second portion of the dry anticoagulant powder 120 deposited on the walls 146 of the second flow channel 142. The second flow channel 142 may also include obstacles or mixing promoters 150 that interfere with the flow path of the blood sample thereby promoting mixing between the blood sample and the second portion of the dry anticoagulant powder 120. After mixing, the second portion 154 of the blood sample and the second portion of the dry anticoagulant powder 120, i.e., a stabilized blood sample, travel to the outlet channel 134.

In other embodiments, other portions of the specimen mixing and transfer device 100 may also include obstacles or mixing promoters 150 that interfere with the flow path of the blood sample thereby promoting mixing between the blood sample and the dry anticoagulant powder 120.

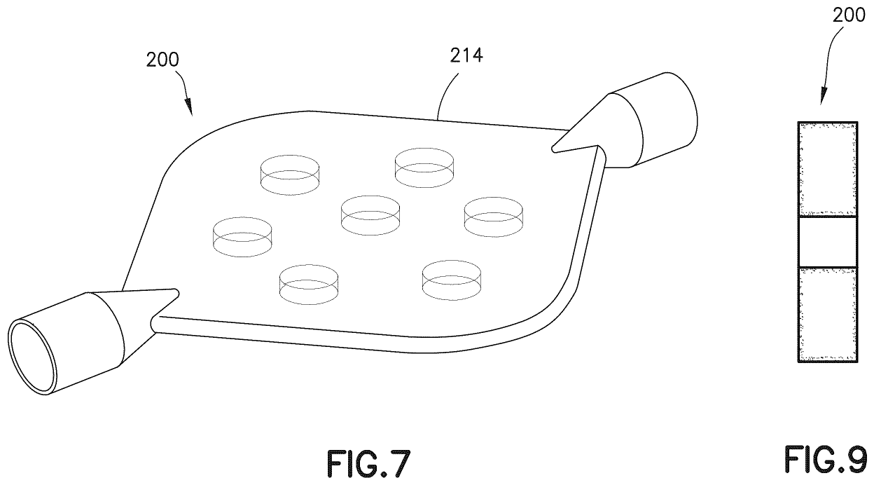

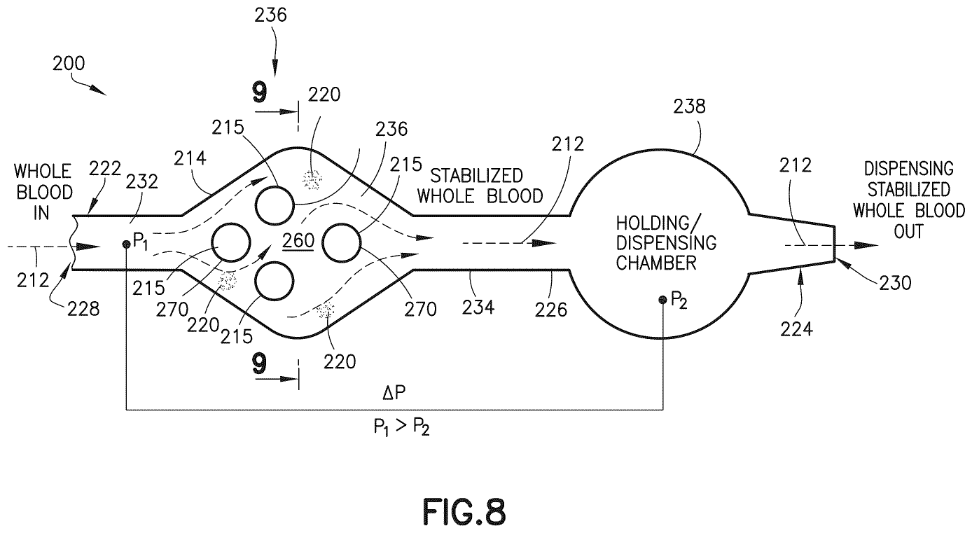

FIGS. 7-10 illustrate other exemplary embodiments of a specimen mixing and transfer device of the present disclosure. Referring to FIGS. 7 and 8, the specimen mixing and transfer device 200 is adapted to receive a sample 212. In some embodiments, the sample 212 may be a blood sample. In one embodiment, the specimen mixing and transfer device 200 includes a housing 214, a dry anticoagulant powder 220 disposed within the housing 214, and a mixing element 215 disposed within the housing 214.

The housing 214 includes a first end 222, a second end 224, and a sidewall 226 extending between the first end 222 and the second end 224. In one embodiment, the first end 222 includes an inlet 228 and the second end 224 includes an outlet 230.

Referring to FIG. 8, in one embodiment, the housing 214 of the specimen mixing and transfer device 200 includes an inlet channel 232 and an outlet channel 234. The inlet channel 232 and the outlet channel 234 are in fluid communication via a flow channel or mixing chamber 236. For example, the inlet channel 232 is in fluid communication with the inlet 228 and the mixing chamber 236; and the outlet channel 234 is in fluid communication with the mixing chamber 236 and the outlet 230. In one embodiment, the dry anticoagulant powder 220 is disposed within the mixing chamber 236 of the housing 214. In one embodiment, the dry anticoagulant powder 220 is deposited on an interior surface 260 of the housing 214.

Referring to FIG. 8, in one embodiment, the housing 214 of the specimen mixing and transfer device 200 includes a dispensing chamber or holding chamber 238. The dispensing chamber 238 may be adjacent to the outlet 230 of the specimen mixing and transfer device 200. For example, the dispensing chamber 238 may be disposed between the mixing chamber 236 and the outlet 230.

In one embodiment, the specimen mixing and transfer device 200 includes a mixing element 215 disposed within the housing 214. In one embodiment, the mixing element 215 includes a plurality of posts 270. For example, the mixing chamber 236 may include a plurality of posts 270 that interfere with the flow path of the blood sample thereby promoting mixing between the blood sample and the dry anticoagulant powder 220.

Referring to FIGS. 7 and 8, the specimen mixing and transfer device 200 is adapted to receive a sample 212 therein via the first end 222. For example, the housing 214 of the specimen mixing and transfer device 200 is adapted to receive a sample 212 therein via the inlet 228. The sample 212 flows into the inlet 228 and to the inlet channel 232. In some embodiments, the sample 212 may be a blood sample.

With the blood sample received within the inlet channel 232, the blood sample flows into the mixing chamber 236. As the blood sample flows into the mixing chamber 236, the blood sample mixes with the dry anticoagulant powder 220 deposited on an interior surface 260 of the housing 214. The mixing chamber 236 may include the plurality of posts 270 that interfere with the flow path of the blood sample thereby promoting mixing between the blood sample and the dry anticoagulant powder 220. After mixing, the blood sample and the dry anticoagulant powder 220, i.e., a stabilized blood sample, travel to the outlet channel 234.

In other embodiments, other portions of the specimen mixing and transfer device 200 may also include mixing elements 215 that interfere with the flow path of the blood sample thereby promoting mixing between the blood sample and the dry anticoagulant powder 220.

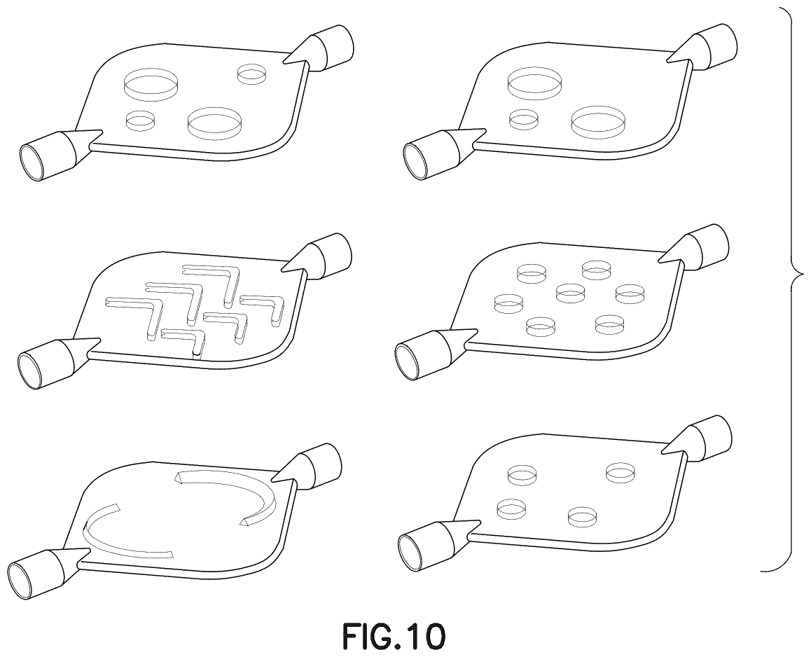

Referring to FIG. 10, alternate embodiments of a specimen mixing and transfer device of the present disclosure are illustrated.

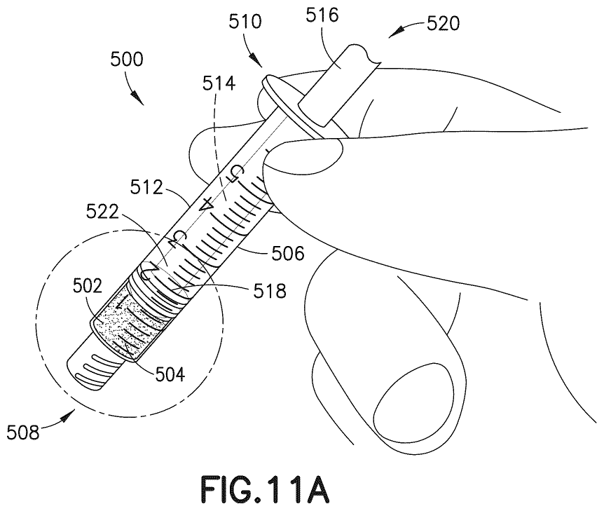

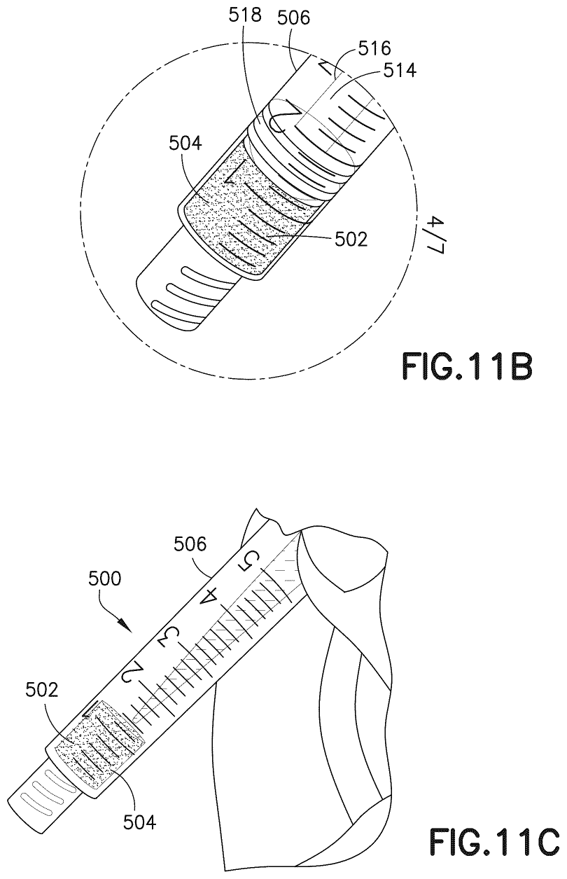

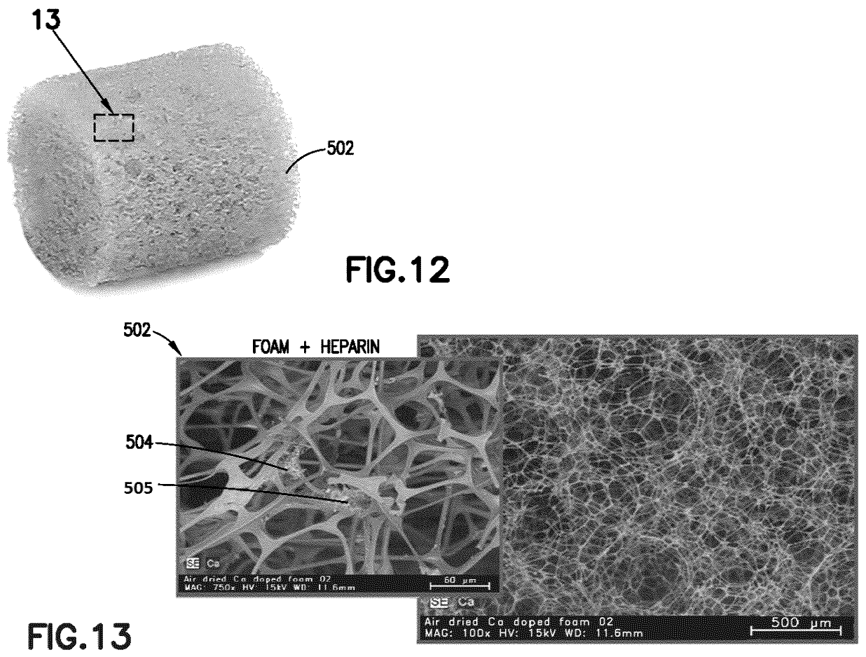

FIGS. 11A-16 illustrate another exemplary embodiment of a material of the present disclosure. The material 502 includes pores 505 and has a dry anticoagulant powder 504 within the pores 505 of the material 502, as described above. In one embodiment, the material 502 is a sponge material. In other embodiments, the material 502 is an open cell foam. In one embodiment, the open cell foam is treated with an anticoagulant, as described in detail above, to form a dry anticoagulant powder 504 finely distributed throughout the pores 505 of the material 502.

In one embodiment, the material 502 is an open cell foam. For example, the material 502 is a soft deformable open cell foam that is inert to blood. In one embodiment, the open cell foam may be a melamine foam, such as Basotect® foam commercially available from BASF. In another embodiment, the open cell foam may consist of a formaldehyde-melamine-sodium bisulfite copolymer. The open cell foam may be a flexible, hydrophilic open cell foam that is resistant to heat and many organic solvents. In one embodiment, the open cell foam may be a sponge material.

Referring to FIGS. 11A-16, the material 502 can be utilized with a syringe assembly 500. The syringe assembly 500 may include an open cell foam material 502 having a dry anticoagulant powder 504 therein. The open cell foam material 502 is disposed within the syringe assembly 500. The anticoagulant can be loaded into the open cell foam material 502 having pores 505, as described above.

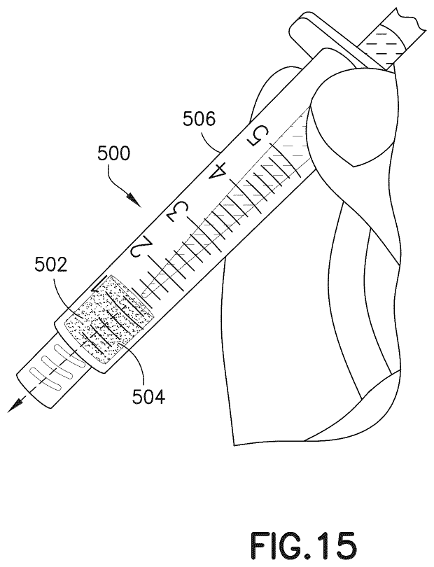

In one embodiment, the syringe assembly 500 includes a syringe barrel 506 having a first end 508, a second end 510, and a sidewall 512 extending therebetween and defining an interior 514. Referring to FIGS. 11A-11C and 15, the open cell foam material 502 is disposed within the interior 514 of the syringe barrel 506.

In one embodiment, the syringe assembly 500 includes a plunger rod 516 and a stopper 518. The plunger rod 516 includes a first end 520 and a second end 522. The stopper 518 is engaged with the second end 522 of the plunger rod 516 and is slidably disposed within the interior 514 of the syringe barrel 506. The stopper 518 is sized relative to the interior 514 of the syringe barrel 506 to provide sealing engagement with the sidewall 512 of the syringe barrel 506.

The open cell foam material 502 is placed in the syringe barrel 506 for mixing and stabilizing blood. The blood gets collected in the syringe barrel 506 with the open cell foam material 502 embedded inside the syringe barrel 506. The stabilized blood can then be dispensed for analysis. In one embodiment, the syringe assembly 500 is an arterial blood gas syringe and the stabilized blood can be dispensed for blood gas analysis.

In one embodiment, the syringe assembly 500 acts as a flow-through chamber for the effective mixing of a blood sample with the dry anticoagulant powder 504 within the open cell foam material 502. In other embodiments, the open cell foam material 502 may contain other dry substances. The effective mixing is achieved by passing the blood sample through the open cell foam material 502 having the dry anticoagulant powder 504 distributed throughout its microstructure.

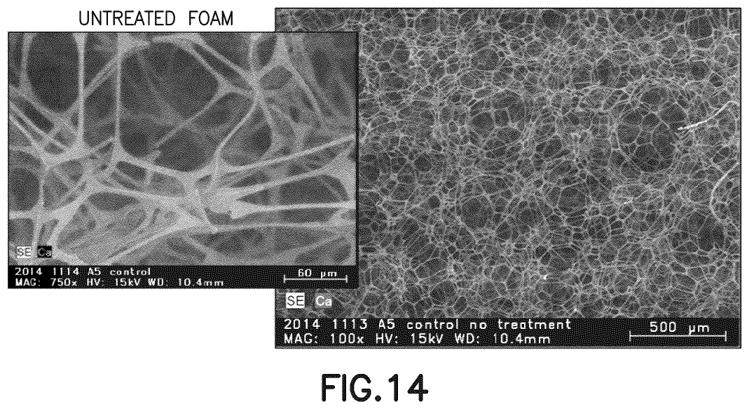

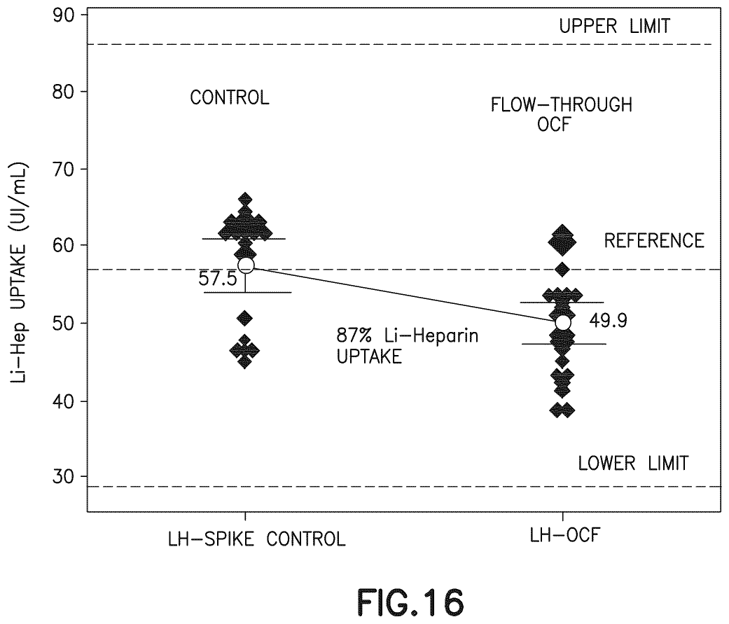

Referring to FIG. 13, a view of the microstructure of the open cell foam material 502 having a dry anticoagulant powder 504 distributed throughout its microstructure is illustrated. Referring to FIG. 14, a view of the microstructure of an untreated foam material 502 is illustrated. Referring to FIG. 16, a graph is illustrated demonstrating the anticoagulant uptake by a blood sample flowing through an open cell foam material having a dry anticoagulant powder distributed throughout its microstructure.

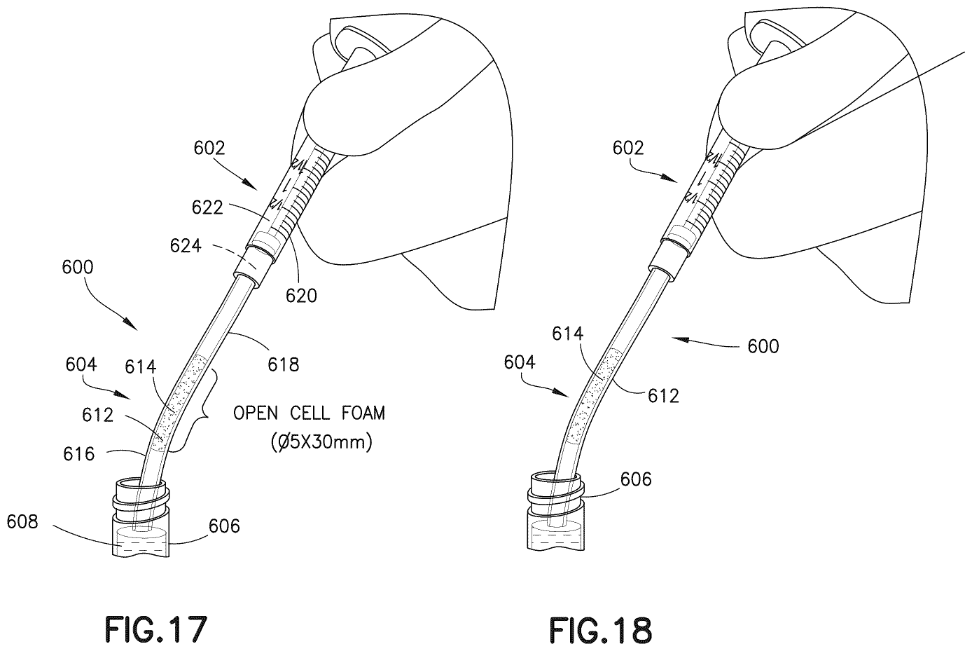

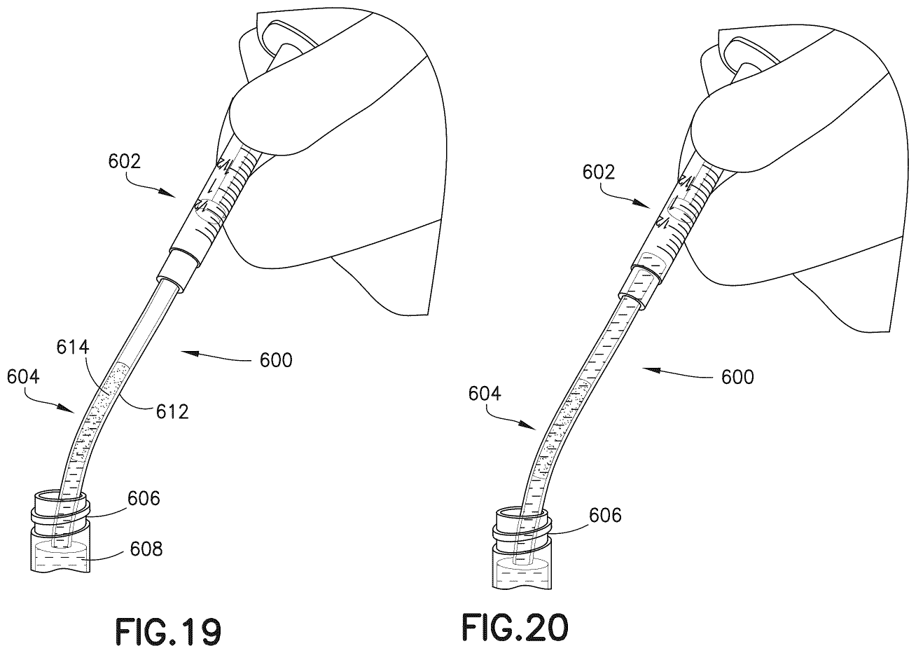

FIGS. 17-20 illustrate an exemplary embodiment of a specimen mixing and transfer system of the present disclosure. Referring to FIGS. 17-20, in one embodiment, a blood transfer system 600 includes a syringe assembly 602, a line 604, and a container 606. In one embodiment, the container 606 contains blood 608.

In one embodiment, the line 604 includes an open cell foam material 612 having a dry anticoagulant powder 614 therein. The anticoagulant can be loaded into the open cell foam material 612 having pores, as described above. The open cell foam material 612 is disposed within the line 604. The line 604 includes a first end 616 and a second end 618.

In one embodiment, the syringe assembly 602 includes a syringe barrel 620 and a sidewall 622 defining an interior 624. Referring to FIGS. 17-20, the line 604 is adapted to place the syringe assembly 602 and the container 606 in fluid communication. For example, the first end 616 of the line 604 can be in fluid communication with the contents of the container 606, and the second end 618 of the line 604 can be in fluid communication with the syringe assembly 602.

The open cell foam material 612 is placed in the line 604 for mixing and stabilizing blood. In one embodiment, the blood 608 is transferred from the container 606 to the syringe barrel 620 via the line 604. For example, a blood sample, e.g., blood 608, passes through the line 604 with the open cell foam material 612 embedded inside the line 604 as the blood gets collected into the syringe barrel 620. In this manner, the blood 608 is stabilized before entering the syringe barrel 620. After the stabilized blood 608 is contained within the syringe barrel 620, the stabilized blood 608 can then be dispensed for analysis.

In one embodiment, the line 604 acts as a flow-through chamber for the effective mixing of a blood sample with the dry anticoagulant powder 614 within the open cell foam material 612. In other embodiments, the open cell foam material 612 may contain other dry substances. The effective mixing is achieved by passing the blood sample through the open cell foam material 612 having the dry anticoagulant powder 614 distributed throughout its microstructure.

The present disclosure provides a material that includes pores and has a dry anticoagulant powder within the pores of the material, as described above. In one embodiment, the material is a sponge material. In other embodiments, the material is an open cell foam. In one embodiment, the open cell foam is treated with an anticoagulant, as described in detail above, to form a dry anticoagulant powder finely distributed throughout the pores of the material.

The present disclosure provides different applications and embodiments of the material. For example, in one embodiment, a specimen mixing and transfer device of the present disclosure is adapted to receive a sample. The specimen mixing and transfer device includes a housing, a material including pores that is disposed within the housing, and a dry anticoagulant powder within the pores of the material. In one embodiment, the material is a sponge material. In other embodiments, the material is an open cell foam. In one embodiment, the open cell foam is treated with an anticoagulant to form a dry anticoagulant powder finely distributed throughout the pores of the material. A blood sample may be received within the specimen mixing and transfer device. The blood sample is exposed to and mixes with the anticoagulant powder while passing through the material.

A specimen mixing and transfer device of the present disclosure offers uniform and passive blood mixing with an anticoagulant under flow-through conditions. A specimen mixing and transfer device of the present disclosure could catch blood clots or other contaminants within the microstructure of the material and prevent them from being dispensed into a diagnostic sample port. A specimen mixing and transfer device of the present disclosure enables a simple, low-cost design for passive flow-through blood stabilization. A specimen mixing and transfer device of the present disclosure enables precisely controlled loading of an anticoagulant into the material by soaking it with an anticoagulant and water solution and then drying the material to form a finely distributed dry anticoagulant powder throughout the pores of the material.

A specimen mixing and transfer device of the present disclosure may provide an effective passive blood mixing solution for applications wherein blood flows through a line. Such a specimen mixing and transfer device is useful for small blood volumes, e.g., less than 50 μL or less than 500 μL, and/or where inertial, e.g., gravity based, forces are ineffective for bulk manual mixing by flipping back and forth a blood collection container such as is required for vacuum tubes.

In other embodiments of the present disclosure, the material can be utilized with a specimen mixing and transfer system or a syringe assembly, as described above.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A syringe assembly adapted to receive a sample, comprising:
a syringe barrel having a first end, a second end, and a sidewall extending therebetween;
a material including pores and disposed within the syringe barrel; and
a dry anticoagulant powder within the pores of the material.

2. The syringe assembly of claim 1, wherein the sample is a blood sample.

3. The syringe assembly of claim 2, wherein the syringe barrel is adapted to receive the blood sample therein via the first end.

4. The syringe assembly of claim 3, wherein, with the blood sample received within the syringe barrel, the blood sample passes through the material thereby effectively mixing the blood sample with the dry anticoagulant powder.

5. The syringe assembly of claim 4, wherein the blood sample dissolves and mixes with the dry anticoagulant powder while passing through the material.

6. The syringe assembly of claim 1, wherein the material is a sponge.

7. The syringe assembly of claim 1, wherein the first end of the syringe barrel includes an inlet and an outlet.

8. The syringe assembly of claim 1, further comprising:
a plunger rod having a first end and a second end; and
a stopper engaged with the second end of the plunger rod, the stopper slidably disposed within the syringe barrel.

9. They syringe assembly of claim 8, wherein the stopper is configured to provide sealing engagement with the sidewall of the syringe barrel.

10. The syringe assembly of claim 2, wherein the syringe assembly is adapted to dispense the blood sample via the first end of the syringe barrel.

11. A syringe assembly adapted to receive a sample, comprising:
a syringe barrel having a first end, a second end, and a sidewall extending therebetween;
a line having a first end and a second end, the second end of the line being in fluid communication with the first end of the syringe barrel;
a material including pores and disposed within the line; and
a dry anticoagulant powder within the pores of the material.

12. The syringe assembly of claim 11, wherein the sample is a blood sample.

13. The syringe assembly of claim 12, wherein the syringe barrel is adapted to receive the blood sample therein via the first end.

14. The syringe assembly of claim 13, wherein, with the blood sample received within the syringe barrel, the blood sample passes through the material thereby effectively mixing the blood sample with the dry anticoagulant powder.

15. The syringe assembly of claim 14, wherein the blood sample dissolves and mixes with the dry anticoagulant powder while passing through the material.

16. The syringe assembly of claim 11, wherein the material is a sponge.

17. The syringe assembly of claim 11, wherein the first end of the line includes an inlet and an outlet.

18. The syringe assembly of claim 11, further comprising:
a plunger rod having a first end and a second end; and
a stopper engaged with the second end of the plunger rod, the stopper slidably disposed within the syringe barrel.

19. They syringe assembly of claim 18, wherein the stopper is configured to provide sealing engagement with the sidewall of the syringe barrel.

20. The syringe assembly of claim 12, wherein the syringe assembly is adapted to dispense the blood sample via the first end of the line.

21. The syringe assembly of claim 12, further comprising:
a container configured to contain the blood sample, the container being in fluid communication with the first end of the line.
